(19) United States
(12) Patent Application Publication
(10) Pub. No.: US 2006/0009914 A1
(43) Pub. Date: Jan. 12, 2006

(54) Detection and identification of nucleic acid, peptide, and protein modifications

(76) Inventors: Narayan Sundararajan, San Francisco, CA (US); Lei Sun, Santa Clara, CA (US); Xing Su, Cupertino, CA (US); Mineo Yamakawa, Campbell, CA (US); Zhang Jingwu, San Jose, CA (US); Selena Chan, San Jose, CA (US); Andrew Berlin, San Jose, CA (US); Tae-Woong Koo, San Francisco, CA (US); Mark Roth, Seattle, WA (US); Phil Gafken, Seattle, WA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/919,699

(22) Filed: Aug. 16, 2004

Related U.S. Application Data
(60) Provisional application No. 60/587,334, filed on Jul. 12, 2004.

(51) Int. Cl. G06F 19/00; G01N 33/48

(52) U.S. Cl. 702/19; 702/20

(57) ABSTRACT

Embodiments of the present invention provide devices and methods for detecting, identifying, distinguishing, and quantifying modifications to nucleic acids, proteins, and peptides using SERS and Raman spectroscopy. Applications of embodiments of the present invention include proteome wide modification profiling and analyses with applications in disease diagnosis, prognosis and drug efficacy studies, enzymatic activity profiling and assays.

### RELATED APPLICATION

This application is a non-provisional application of, and claims the benefit of the earlier filed U.S. Provisional Ser. No. 60/587,334, filed on Jul. 12, 2004, currently pending.

### BACKGROUND OF THE INVENTION

1. Field of the Invention

The embodiments of the present invention relate generally to the use of Raman spectroscopy for detecting, distinguishing, quantifying, and identifying modifications to nucleic acids, peptides, and proteins.

2. Background Information

Post-translational modifications of proteins are said to play an important role in the biological activity of proteins. Hence, understanding whether a protein is modified or not and the type and nature of the modification would be very beneficial to understanding cell cycles and processes and the role of proteins in them. Currently, mass spectroscopy (MS) and specific antibodies tailored to particular modifications of the amino acids in a peptide sequence are the two commonly used methods.

Post-translational modifications (PTMs) are chemical processing events that cleave or add modifying groups to proteins for the purpose of modulating precise regulatory functions in a cell. Over 200 different types of PTMs have been described (1) and PTMs such as acetylation (2), methylation (3), phosphorylation (4), ubiquitination (5), and others play key roles in the regulation of gene expression, protein turnover, signaling cascades, intracellular trafficking, and cellular structure.

The biological importance of PTMs has been widely recognized, and MS has been a favored approach for proteome-wide PTM profiling due to its sensitivity for measuring and locating molecular weight changes in proteins and peptides (6-8). However, some modifications such as acetylation/trimethylation of lysine (both have nominal mass increases of 42 Da) and phosphorylation/sulfation of tyrosine (both have nominal mass increases of 80 Da) require expensive, high-resolution mass spectrometers (9, 10) or require mass spectrometry analysis schemes that are not conducive to high-throughput analyses. Also, modifications such as phosphorylation, sulfation, and glycosylation are unstable during tandem mass spectrometry experiments making identification and positional information difficult, if not impossible, to obtain. In few cases, quantification of protein expression and modifications using mass spectrometry has been performed using stable isotope labeling techniques (11, 12).

### DESCRIPTION OF THE FIGURES

FIG. 1 is a schematic diagram illustrating steps for protein profiling using SERS or Raman spectroscopy. Optionally, the protein profiling may also include mass spectrometry.

FIG. 2 contains two schematics, each illustrating a use of SERS to detect peptide modifications. In the top schematic, a substrate containing an array having a multiplexity of peptides at different locations is allowed to interact with a biosample (containing, for example, enzymes or cell lysates), and SERS is performed before and after the interaction to detect differences. In the bottom schematic, a peptide array is made from a digested set of proteins or biofluids deposited on a substrate, selected enzymes are reacted with the peptides of the array, and SERS is performed before and after the enzymatic interaction to detect peptide modifications.

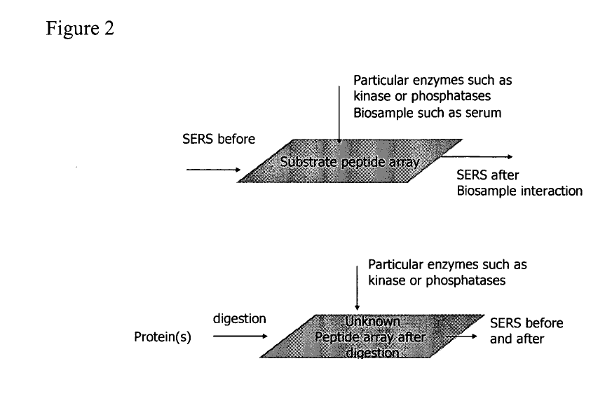

FIG. 3 shows the SERS spectra of an unmodified peptide (sequence: ⁹KSTGGKAPR) with notations regarding the chemical bonding information that can be derived from the peaks (spectra taken at a peptide concentration of 9 ng/μl).

FIG. 4 shows SERS spectra of unmodified and modified peptides (K9 peptide of the histone H3.3 of drosophila); KSTGGKAPR(H3), (K-trimethylated)STGGKAPR(H3-3Me), (K-acetylated)STGGKAPR(H3-Ac) (spectra were taken a concentration of 9 ng/μl each). It can be seen that the spectral signatures of the peptides differ based on the modification of a single amino acid. The spectra were arbitrarily offset along the y-axis for clarity.

FIG. 5 shows the detection of very low concentrations of trimethylated peptide. The lowest concentration that was detected was about 9 pg/μl which corresponds to 1 zeptomole of peptide in the collection volume (assuming a laser spot size of 5 μm×5 μm×5 μm leading to a collection volume of 125 femtoliters). The spectra were arbitrarily offset along the y-axis for clarity. Arrows point to strong spectral features that are clearly present at all concentrations.

FIGS. 6A and B illustrate the position dependence effect on the SERS spectra for two different modifications: trimethylation and phosphorylation. FIG. 6A: Top—SERS spectra of peptide trimethylated at the lysine inside the peptide chain (KSTGGK(trimethylated)APR); Bottom—SERS spectra of same peptide trimethylated at the N-terminus lysine (K(trimethylated)STGGKAPR (spectra were taken at concentrations of 9 ng/μL and arbitrarily offset along the y-axis). FIG. 6B (Phosphorylation position dependence): Top—Phosphorylation at the Threonine (KST(phosphorylated)GGKAPR); Bottom—Phosphorylation at the Serine (KS(phosphorylated)TGGKAPR) (spectra were taken at concentrations of 90 ng/μL and arbitrarily offset along the y-axis).

FIGS. 7A provides Raman spectra obtained from a 50:50 concentration mixture of the two modified acetylated (K(acetylated)STGGKAPR) and trimethylated (K(trimethylated)STGGKAPR) peptides showing the presence of peaks corresponding to both the modifications. FIG. 7B graphically illustrates the ratio of peak heights corresponding to acetylation and trimethylation exhibiting a linear trend with increasing acetylated peptide content. The Y-axis represents the ratio of intensities of peaks at 628 cm⁻¹ and 744 cm⁻¹ from the SERS spectra of different volume % mixtures. The X-axis represents the % concentration of 9-acetylated peptide P-9Ac in the mixture.

FIG. 8 shows SERS spectra of acetylated modified peptide (K(acetylated)STGGKAPR) as a function of incubation time of colloidal silver+peptide before LiCl addition and SERS spectroscopy.

FIG. 9 shows SERS spectra of peptide P-9Ac (⁹K_{ac}STGGKAPR) at different incubation times of sample with the colloidal silver solution. 80 μl of silver solution (1:2 diluted in water) was mixed with 10 μl of the peptide (100 ng/μl) and incubated at room temperature for 0-20 min. 20 μl of lithium chloride solution (0.5M in DI water) was added to the above solution and SERS spectra were accumulated immediately after LiCl addition by dropping the solution onto an aluminum substrate.

FIG. 10 shows SERS spectra of separated fractions from HPLC obtained from digested Histone H3 from Drosophila. SERS spectra indicate signature peptide peaks.

FIG. 11 shows SERS spectra of a mixture of unmodified and phosphorylated peptide (KST(phosphorylated)GGKAPR) at different ratios. Peak height at 628 cm⁻¹ (not normalized) corresponding to phosphorylation is plotted against vol. % of phosphorylated peptide. An almost linear trend is observed.

FIG. 12 shows the ratio of intensities of peaks at 744 cm⁻¹ (trimethyl) and 1655 cm⁻¹ (Amide I) wave numbers are plotted for the two peptides P-9Me3 and P-14Me3. 50 spectra (accumulation time=1 s) were collected for each peptide and the peak intensities at 744 cm⁻¹ and 911 cm⁻¹ were calculated for each spectra and their ratios taken. Average for the ratio of the intensities for the peptides P-9Me3 and P-14Me3 were 2.499 and 1.644 with standard deviations of 0.0586 and 0.0437 respectively.

FIG. 13 shows the ratio of intensities of peaks at 628 cm⁻¹ and 1655 cm⁻¹ wave numbers plotted for different concentration ratio mixtures of the two peptides, unmodified P and phosphorylated P-11P. 50 spectra (accumulation time=1 s) were collected for each mixture and the peak intensities at 628 cm⁻¹ and 1655 cm⁻¹ were calculated for each spectra. Plot below shows the ratios of intensities of the two peaks plotted against the % concentration of the phosphorylated peptide in the mixture.

FIG. 14A shows a raw sample spectrum of the unmodified peptide P. Background from the spectra was subtracted by fitting an arbitrary linear baseline FIG. 14B shows how intensities of peaks were calculated directly from the raw spectra by calculating the distance between the apex of the peak area and the midpoint of the base points of the peak area.

FIG. 15 shows a schematic of a Raman spectrometer setup that can be used for SERS measurements.

### DETAILED DESCRIPTION OF THE INVENTION

Embodiments of the present invention provide devices and methods for identifying, distinguishing, and quantifying modifications to nucleic acids, proteins, and peptides using SERS and Raman spectroscopy. Applications of embodiments of the present invention include proteome wide modification profiling and analyses with applications in disease diagnosis, prognosis and drug efficacy studies, enzymatic activity profiling and assays.

A variety of modifications are possible on amino acids in a peptide or a protein sequence and the present invention is not limited in the types of modifications that can be detected. (See e.g., “Proteomic analysis of post-translational modifications”, Mann et al., Nature Biotechnology, 21:255 (2003)). Embodiments of the present invention provide the ability to detect various modifications of the amino acids in a peptide sequence at very low concentrations, and also to distinguish, identify and quantify them based on spectral signatures even if their masses are very similar. For example, embodiments of the present invention provide the ability to detect acetyl and trimethyl modifications on a lysine amino acid that differ by about 0.02 amu. The present invention also provides methods that provide positional information for labile modifications such as, for example, serine and threonine phosphorylation.

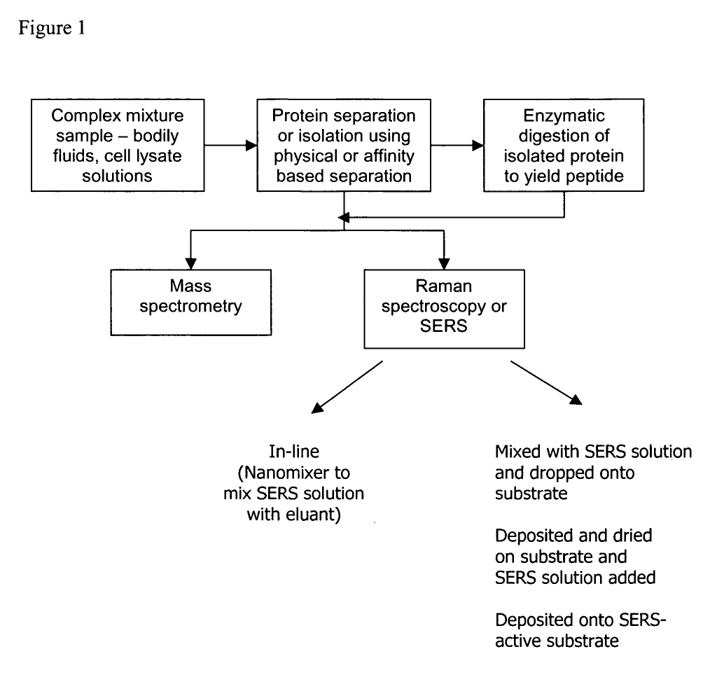

SERS and Raman analysis can be used stand-alone or in conjunction with Mass spectrometry (for example, ESI or MALDI) to obtain modification or protein profiles of different biofluids such as serum after physical or affinity-based (using antibody-based) separation for applications such as disease diagnosis and prognosis, and drug efficacy applications. FIG. 1 shows a schematic for protein profiling using surface enhanced Raman spectroscopy stand-alone or in conjunction with Mass spectroscopy.

In doing SERS or Raman spectroscopy, different formats can be used for analyzing the eluants from the separation devices used for simplification of complex mixtures. In one embodiment, the eluants can be deposited onto a SERS-active substrate or dried onto a substrate and SERS colloidal silver solution added before detection. Another format is to mix the silver colloidal solution with the eluants in the fluidic format (optionally, on chip) and perform the detection inline as the eluants are flowing through the laser detection volume. In additional embodiments, some or all of these steps are performed using microfluidics.

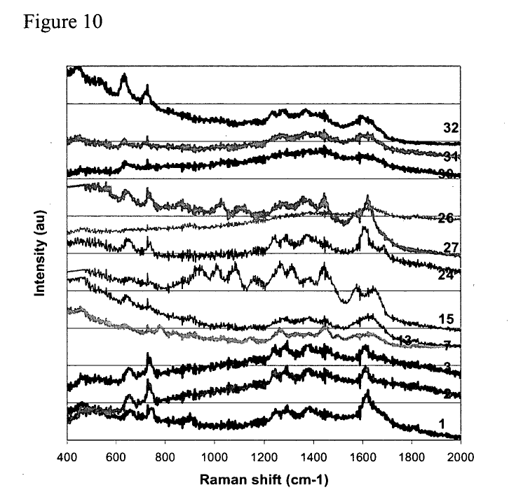

FIG. 10 shows the SERS spectra obtained from fractions separated by HPLC of Histone H3 protein digested by Arg-C protease.

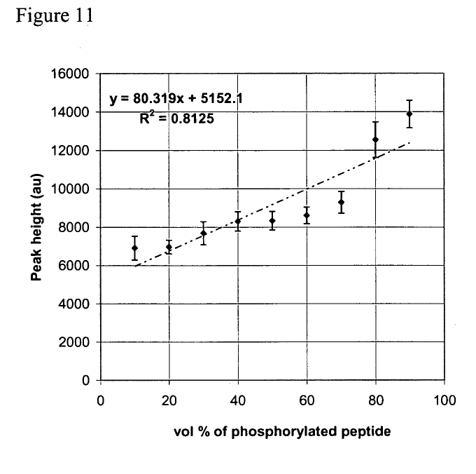

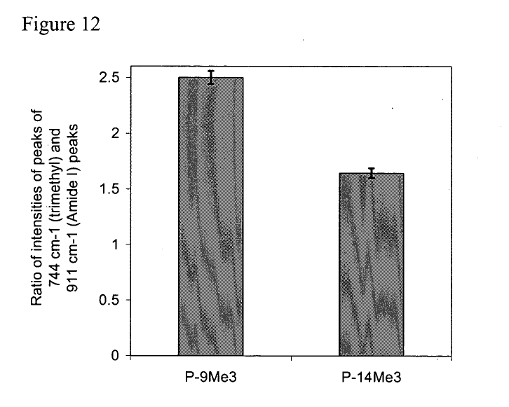

FIG. 11 shows the quantification information obtained from mixing an unmodified and a phosphorylated peptide at different vol % and correlating the intensity of the peak height corresponding to phosphorylation that is not present in the unmodified form to the % of phosphorylated peptide. In one embodiment of the present invention enzymatic activity assays such as phosphotase, kinase, acetylase, and deacetylase assays etc. are performed using SERS spectroscopy. For example, FIG. 12 shows a schematic illustrating two methods for different types of enzymatic activity profiling. A known peptide array is synthesized using photolithography techniques and is used as the substrate for testing the activity (yes or no type assay or quantification) of different types of enzymes or lysates. SERS is performed before and after the enzymatic or lysate activity on the substrate peptide array to understand the activity of particular enzymes on particular substrate peptides or lysates on particular peptides. In a second example, the array is comprised of unknown peptides obtained from digestion of proteins or biofluids. The activity of particular enzymes is determined and profiles are generated from different biofluids. In additional embodiments, SERS is used for disease diagnosis and drug efficacy screening. In a further embodiment, SERS is used as a screening tool for drug candidate molecules by identifying or profiling enzymatic activity.

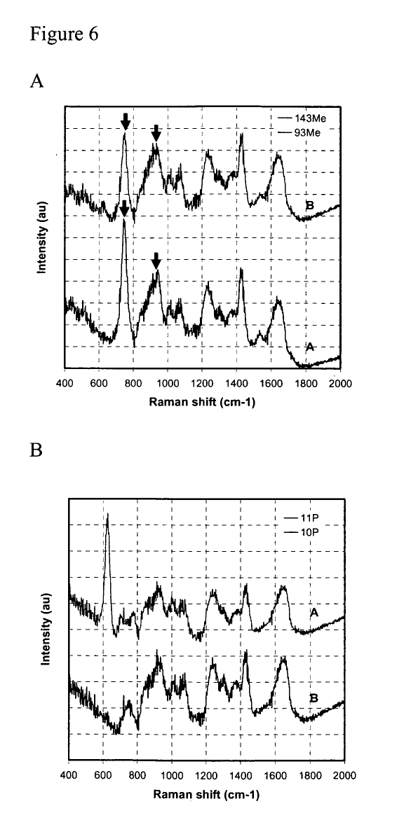

It was demonstrated that SERS is effective in obtaining position information for modifications such as trimethylation and phosphorylation within a peptide. FIG. 6A compares the SERS spectra of trimethylated modified peptides with the trimethylation modification at either the lysine at the 9 amino-acid position (peptide P-9Me3) or at the lysine at the 14 amino-acid position (peptide P-14Me3). It is apparent from the SERS spectra that the intensity of the peak at 744 cm⁻¹ is reduced in the peptide P-14Me3 compared to the peptide P-9Me3 while the intensity of the peak at 1655 cm⁻¹ does not change significantly in the peptides. This is believed to be because the mechanism of SERS enhancement is attributed to both electromagnetic (27, 28) and chemical effects (29) wherein chemical interactions between the molecules and the metal surfaces not only increase the scattering cross-section of the molecules but also provide the distinct advantage of discerning subtle chemical and conformational changes of molecules. Adsorption and orientation of the molecules onto the silver nanoparticles (30) also play a role in the SERS enhancement. Since the surface of the silver colloidal nanoparticles used in the SERS experiments is negatively charged, it is likely that both the positively charged N-terminus of the peptide and the trimethyl modification adsorb to the silver nanoparticle surface. Consequently, in the case of the peptide P-9Me3 where the trimethyl modification moiety remains close to the metal surface, the peak at 744 cm⁻¹ is strongly enhanced. Whereas, in the peptide P-14Me3, where the trimethyl modification moiety is further away from the silver surface, the intensity of the peak at 744 cm⁻¹ drops relative to the other peaks in the spectra (see FIG. 12 where the ratio of the intensities of peak corresponding to the trimethyl modification (at 744 cm⁻¹) and Amide I (at 1655 cm⁻¹) is plotted for peptides P-9Me3 and P-14Me3. Data analysis was performed using 50 spectra with accumulation times of 1 s for each peptide).

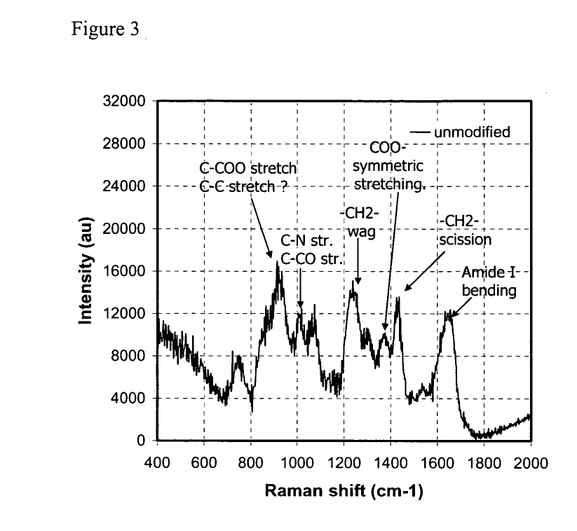

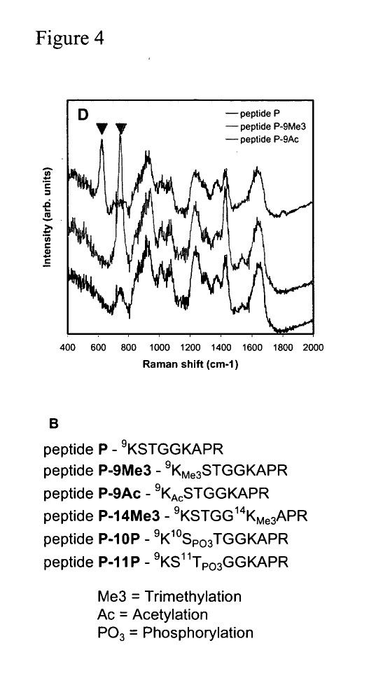

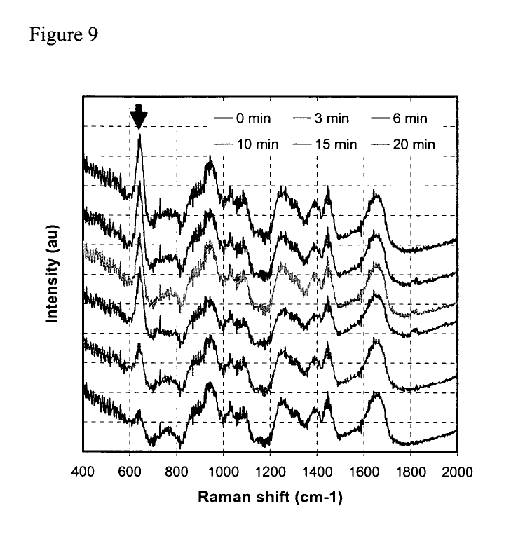

In one embodiment, the present invention provides the ability to detect the presence of post-translational modifications of nearly identical mass on peptides using SERS. We chose part of the N-terminal tail of histone H3 (⁹KSTGGKAPR) as a model substrate peptide because the lysines at the amino-acid positions 9 and 14 in this peptide are frequently targeted for modifications such as acetylation and methylation (17-19) and the serine and threonine at amino acid positions 10 and 11 respectively are targeted for phosphorylation (20, 21). These modifications are known to have major effects on the histone-histone as well as the histone-regulatory protein interactions (2, 3, 20-23). FIG. 3 shows the SERS spectrum of the unmodified peptide. The peaks in the SERS spectrum can be assigned to different vibrational bands within the peptide (24, 25). Particularly strong peaks can be observed at 919 cm⁻¹ (C—COO⁻), 1250 cm⁻¹ (CH₂ wag), 1436 cm⁻¹ (CH₂ scission) and 1655 cm⁻¹ (Amide I). SERS spectra of 9-trimethylated (peptide P-9Me3) and 9-acetylated (peptide P-9Ac) peptides were compared to that of the unmodified peptide (FIG. 4). Clear peaks were observed in the SERS spectra of both the trimethylated and acetylated peptides that were absent from the spectrum of the unmodified peptide (arrowheads in FIG. 4). Even though the mass difference between these modifications is only 0.03639 amu, we could distinguish them from one another (FIG. 4). A very strong peak is observed at a wave-number of 744 cm⁻¹ for the 9-trimethylated peptide P-9Me3, due to the trimethyl modification (CH₃ terminal rocking) of the lysine. The high signal intensity of this peak can be attributed to the strong interaction between the positively charged N-terminus and the trimethyl ammonium side chain with the negatively charged silver nanoparticles (the surface charge density (Zeta potential) for the silver colloidal particles were measured using a Zetasizer (Zetasizer Nano, Malvern) and found to be about 62±3 mV). In the case of the 9-acetylated peptide P-9Ac, a strong peak is observed at a wave-number of 628 cm⁻¹ that can be assigned to the side chain O═C—N bending resulting from the acetyl modification. (It was also found that the intensity of this peak was dependent on the incubation time of the sample with the silver nanoparticles before the addition of lithium chloride for aggregation. See FIG. 9 for SERS spectra of peptide P-9Ac at different incubation times.)

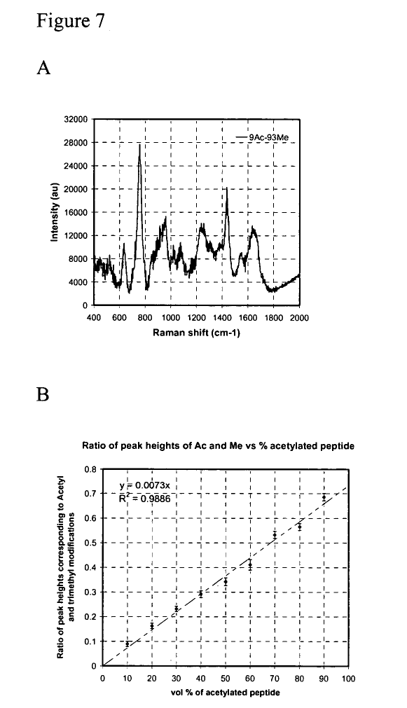

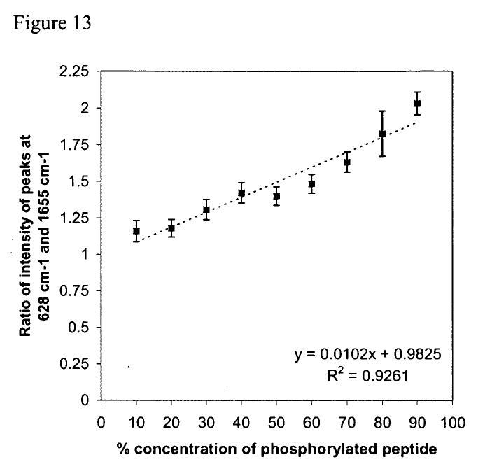

We also explored the ability of SERS to detect two peptides with different modifications in a mixture. FIG. 7A shows the SERS spectra of a mixture of 9-trimethylated peptide, P-9Me3 and 9-acetylated peptide, P-9Ac. Unique peaks at 744 cm⁻¹ and 628 cm⁻¹ that were present in the SERS spectra of the peptides P-9Me3 and P-9Ac are also clearly visible in the spectra of the mixture indicating the presence of both the 9-acetylated and the 9-trimethylated peptides. In addition to detecting the presence of acetylation and trimethylation in mixtures of peptides, we attempted quantification of each type of modification. SERS was performed on mixtures of different concentrations of 9-acetylated and 9-trimethylated peptides, P-9Me3 and P-9Ac. FIG. 7B shows the graph of the ratio of the intensities at 628 cm⁻¹ (corresponding to the acetyl modification) and 744 cm⁻¹ (corresponding to the trimethyl modification) plotted against % concentration of 9-acetylated peptide in the mixture exhibiting a linear trend. SERS spectra allowed us to determine the amount of phosphorylated peptide in a mixture of unmodified (peptide P) and phosphorylated (peptide P-11P) peptides (see FIG. 13 where the ratio of the intensities of peaks at 628 cm⁻¹ and 1655 cm⁻¹ for different mixtures of unmodified peptide P and phosphorylated peptide P-11IP is plotted against the % concentration of phosphorylated peptide P-11P. Data analysis was performed using 50 spectra with accumulation times of 1 s for each peptide. Phosphorylation was detected at % concentration of <10%. This is important as the stoichiometry of phosphorylation is known to be low for specific amino acid sites. This quantification ability particularly lends itself to performing enzymatic activity assays such as kinase and phosphatase assays.)

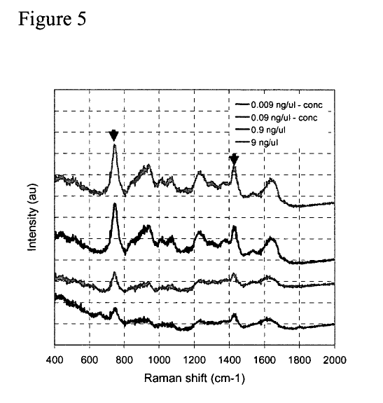

Using SERS, zeptomoles of the trimethylated modified peptide P-9Me3 were detected. This is useful because the stoichiometry of post-translational modifications can be very low. FIG. 5 shows the spectra of the 9-trimethylated peptide P-9Me3 at different concentrations over three orders of magnitude ranging from 9 ng/μl to 9 pg/μl. Concentrations down to 9 pg/μl, which corresponds to about 10 fmol/μl, exhibit the same features (strong peaks at 744 cm⁻¹ and 1436 cm⁻¹) observed in spectra from higher concentrations of the 9-trimethylated peptide P-9Me3. A concentration of 9 pg/μl corresponds to about 20 zeptomoles of the 9-trimethylated peptide P-9Me3 in the collection volume of the laser beam (the collection volume of the laser illumination spot was estimated to be about 2.5×2.5×200 μm).

In a further embodiment, SERS is used for the detection and analysis of labile PTMs, such as, for example, phosphorylation. While the relative ratio of peaks is altered by trimethylation at different positions as shown in FIG. 6A, phosphorylation at different amino acid positions is marked by spectral signature changes. FIG. 6B illustrates the spectral differences between peptides phosphorylated at serine-10 (peptide P-10P, ⁹K¹⁰S_{PO3}TGGKAPR) and threonine-11 (peptide 11-⁹KS¹¹Tp_{PO3}GGKAPR). A strong peak at 628 cm⁻¹ is present only in the case of the peptide P-11P and not in the peptide P-10P. It has been discovered that specific different functional groups at the oligo termini enhance specific peaks in the SERS spectra. In the case of phosphorylation modification, the spectral differences are likely due to the negatively charged phosphate groups affecting the adsorption and orientation of the peptides onto the silver nanoparticles. These results indicate the SERS platform can not only distinguish between peptides modified at different amino acid positions but also identify the precise position of those modifications with single amino acid resolution.

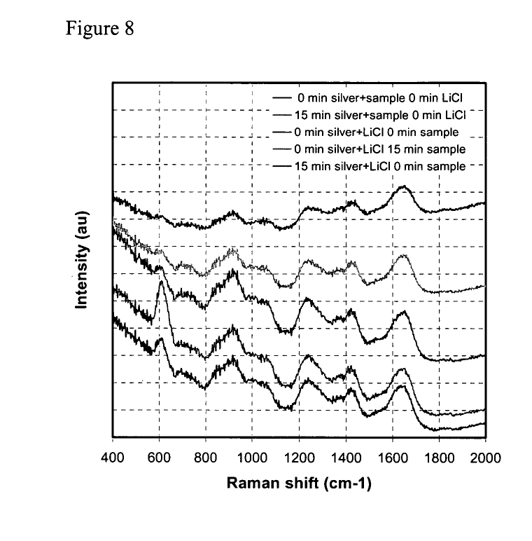

FIGS. 8 and 9 show the effect of some of the factors involved in obtaining a SERS spectra, such as the addition sequence of the SERS cocktail and the incubation time dependence on the SERS spectra of one modified peptide such as the acetylated peptide (K(Acetylated)STGGKAPR). Additionally, the pH, ionic strength, and surface properties of the SERS substrate affect the spectrum obtained. In some embodiments of the present invention, the pH was controlled to have a delta less than about 0.5 pH and ionic strength was controlled, e.g., about 20-300. In addition to the potential effects of pH changes on the spectroscopic and biochemical measurements, the effects of buffering capacity, which are dependent on the concentrations and the types of buffers, also play a role in determining the spectra obtained. For example, performing SERS in acidic condition (such as directly from an HPLC eluent of 0.1% TFA in ACN) increases the signal variations from chemical bonds that are closer to the N-terminal; while performing SERS using Ag particles coated with hydrophobic compounds (such as alkyl-thiol) magnifies the signal change from hydrophobic amino acid such as Tyrosine. Also, the use of complexing agents such as divalent salts (Ca²⁺) for masking or complexing with negative charges on a phosphorylation modification can help in bringing the biomolecule closer to the SERS substrate thereby increasing the ability to distinguish the modified peptide from an unmodified one.

SERS measurements can be performed on a variety of Raman instruments that are known in the art.

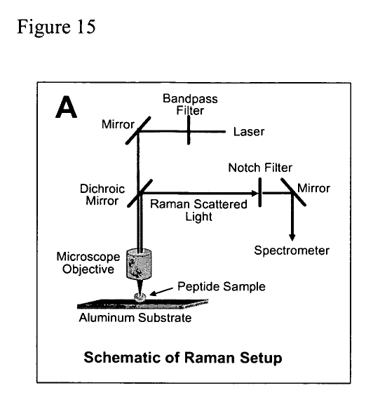

FIG. 15 shows a schematic of a Raman spectrometer setup was used for the SERS measurements discussed herein. The system consisted of a titanium:sapphire laser operating at 785 nm with power levels of about 750 mW, and a 20× microscope objective to focus the laser spot onto the sample plane. The Raman-scattered light was back-collected using a combination of optical components, such as a dichroic filter and a holographic notch filter, and imaged onto the slit of the spectrophotometer connected to a thermo-electrically cooled charge-coupled device (CCD) detector. SERS spectra were obtained from an aqueous solution of the sample peptide on an aluminum substrate as described in the Example 1.

### EXAMPLE 1

Colloidal Silver Preparation

Colloidal silver suspension was prepared by citrate reduction of silver nitrate as described in Lee and Meisel (31). The suspension had a final silver concentration of 1.00 mM. Its zeta potential, after diluting 20 times with DI water, was found to be 62±3 mV (Zetasizer Nano, Malvern).

Peptide Synthesis

Peptides with and without modifications were synthesized using Solid Phase Peptide Synthesis (SPPS) method with standard Fmoc/t-buty/trityl protection chemistries to build up a full-length peptide chain. The starting amino acid was bound to a solid resin support (usually polystyrene) and its alpha amino group was chemically “blocked” with the Fmoc protecting group. Reactive side-chains were blocked with either t-Butyl or Trityl groups. The alpha-amino Fmoc protecting group was removed and an incoming amino acid (which was chemically activated on its carboxyl terminus to form an active ester) condenses to form a peptide bond. The process was repeated until the full-length product was obtained. The resin-bound peptide was then treated with trifluoroacetic acid (TFA) to remove the side-chain protecting groups and cleave the peptide from the polystyrene resin. Peptides were then precipitated out of solution with MTBE (ether) and lyophilized to dryness. For synthesis of modified peptides, trimethylated amino acid analogs were bought from Bachem in Switzerland, phospho-amino acids and acetyl-lysine were purchased from Nova Biochem in San Diego, Calif. Reverse-phase HPLC was utilized to purify and separate the target peptide from a crude mixture. MALDI-TOF Mass Spectrometry was used to determine the peptide's mass and compare with the expected peptide mass to confirm fidelity of the synthesis and purity of the product.

SERS Measurements

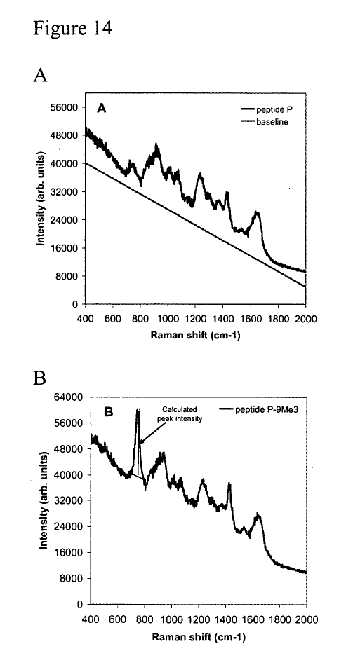

Peptides lyophilized after synthesis were resuspended in DI water at a concentration of 1 μg/μl and diluted to various sample concentrations. The stock solution of the synthesized colloidal silver, with a final silver concentration of 1.00 mM, was diluted 1 part to 2 parts in volume of DI water. Typically, 10 μl of the peptide solution was incubated with 80 μl of the diluted silver solution for 15 min. 20 ill of 0.5 M LiCl solution was added after the incubation and the solution was mixed thoroughly and dropped onto an aluminum tray for immediate SERS measurements. The laser was focused inside the sample droplet and 50-100 spectra were collected for each peptide sample. Typical collection time of each spectrum was 1 sec. A raw sample spectrum of the unmodified peptide P is shown in FIG. 14A. Background from the spectra was subtracted by fitting an arbitrary linear baseline (also shown in FIG. 14A). Intensities of the peaks were calculated directly from the raw spectra by calculating the distance between the apex of the peak area and the midpoint of the base points of the peak area (FIG. 14B).

### REFERENCES

1. R. G. Krishna, F. Wold, in PROTEINS: Analysis & Design. (Academic Press, San Diego, 1998) pp. 121.
2. S. K. Kurdistani, S. Tavazoie, M. Grunstein, Cell 117, 721-733 (2004).
3. T. Kouzarides, Curr Opin Genet Dev 12, 198-209 (2002).
4. P. Cohen, Trends Biochem. Sci. 25, 596-601 (2000).
5. P. Tyers, P. Jorgensen, Curr. Opin. Genet. Dev. 10, 54-64 (2000).
6. M. Mann, O. N. Jensen, Nature 21, 255 (2003).
7. R. E. Schweppe, C. E. Haydon, T. S. Lewis, K. A. Resing, N. G. Ahn, Acc. Chem. Res. 36, 453-461 (2003).
8. R. Aebersold, D. R. Goodlett, Chem. Rev. 101, 269-295 (2001).
9. A. G. Marshall, C. L. Hendrickson, G. S. Jackson, Mass Spectrom. Rev. 17, 1-35 (1998).
10. S. E. Martin, J. Shabanowitz, D. F. Hunt, J. A. Marol, Anal. Chem. 72, 4266-4274 (2000).
11. S. P. Gygi et al., Nature Biotechnology 17, 994 (1999).
12. Zhang X, Jin Q K, Carr S A, A. R S., Rapid Commun Mass Spectrom. 16, 2325-32 (2002).
13. E. B. Hanlon et al., Phys. Med. Biol. 45, R1-R59 (2000).
14. D. Zhang et al., Analytical Chemistry 75, 5703-5709 (2003).
15. K. Kneipp et al., Phys. Rev. E 57, R6281 (1998).
16. K. Kneipp, H. Kneipp, I. Itzkan, R. R. Dasari, M. S. Feld, Journal of Physics C14, R597 (2002).
17. Ahmad K, H. S, Mol Cell 9, 1191-1200 (2002).
18. E. McKittrick, P. R. Gafken, K. Ahmad, S. Henikoff, PNAS 101, 1525-1530 (2004).
19. K. Zhang et al., Analytical Biochemistry 306, 259-269 (2002).
20. B. D. Strahl, C. D. Allis, Nature 403, 41-45 (2000).
21. S. J. Nowak, V. G. Corces, Trends in Genetics 20, 214-220 (2004).
22. S. L. Berger, Curr Opin GenetDev 12, 142-148 (2002).
23. Tamaru H et al., Nat Genet. 34, 75-79 (May 2003, 2003).
24. S. Stewart, P. M. Fredericks, Spectrochimica Acta Part A 55, 1615-1640 (1999).
25. W. Herrebout, K. Clou, H. O. Desseyn, N. Blaton, Spectrochimica Acta Part A 59, 47-59 (2003).
26. S. C. Galasinski, D. F. Louie, K. K. Gloor, K. A. Resing, N. G. Ahn, JBC 277, 2579-2588 (2002).
27. H. Xu, J. Aizpurua, M. Kall, P. Apell, Physical Review E 62, 4318-4324 (2000).
28. M. Kerker, Acc. Chem. Res. 17, 271-277 (1984).
29. A. Campion, P. Kambhampati, Chemical Society Review 27, 241-249 (1998).
30. L. Xu, Y. Fang, Spectroscopy 18, 26-31 (2003).
31. P. C. Lee, D. J. Meisel, Phys. Chem. 86, 3391 (1982).

What is claimed is:

1) A method for detecting a modification state of a protein, peptide, or nucleic acid, comprising obtaining a surface enhanced Raman spectrum of the protein, peptide, or nucleic acid and analyzing the spectrum to ascertain the modification state of the protein, peptide, or nucleic acid.

2) A method for detecting a modification state of at least one protein comprising, obtaining a sample containing a target protein, isolating a protein fraction from the sample containing the target protein, digesting the protein fraction to create peptide fragments, obtaining a SERS spectrum of the peptide fragments, and determining a modification state of at least one protein from the data contained in the SERS spectrum.
